(19) United States
(12) Patent Application Publication
(10) Pub. No.: US 2025/0248729 A1
(43) Pub. Date: Aug. 7, 2025

(54) ENDOVASCULAR DEVICES WITH EXTENDIBLE SHAVER

(71) Applicant: Bard Peripheral Vascular, Inc., Franklin Lakes, NJ (US)

(72) Inventor: Matthew R. CASIRARO, Tempe, AZ (US)

(73) Assignee: Bard Peripheral Vascular, Inc., Franklin Lakes, NJ (US)

(21) Appl. No.: 18/857,112

(22) PCT Filed: Apr. 22, 2022
(86) PCT No.: PCT/US2022/025950

§ 371 Date: Oct. 15, 2024

(51) Int. Cl. A61B 17/32; A61B 17/00

(52) U.S. Cl. CPC A61B 17/32002; A61B 2017/00778; A61B 2017/00867; A61B 2017/320024

(57) ABSTRACT

An endovascular device is described herein. The endovascular device may be utilized for a variety of treatments, for example, treating May Thurner Syndrome. The endovascular device includes a catheter, a needle extending within a lumen of the catheter, and a shaving mechanism at least partially arranged within a lumen of the needle. The needle is movable within the catheter, and distal movement of the needle advances a distal needle tip of the needle out of the catheter and proximal movement of the needle retracts the distal needle tip within the lumen of the catheter. The shaving mechanism includes an abrasive burr positioned proximate the distal needle tip, wherein the abrasive burr is rotatable relative to the needle upon activation of the shaving mechanism.

### TECHNICAL FIELD

The present disclosure relates generally endovascular devices, more particularly, to a device an endovascular device with extendible shaver.

### BACKGROUND

May-Thurner syndrome is extrinsic venous compression by the arterial system against bony structures in the ileocecal venous territory, most commonly of the left common iliac vein by the right common iliac artery. Since the vein is compressed, this becomes an area of constriction and is an area that can lead to thrombus aggregation due to the constriction. Recently, May-Thurner syndrome has been treated with a venous stent to open up the vein. However, this does leave a permanent implant behind.

May-Thurner syndrome (also known as iliac vein compression syndrome) affects two blood vessels that go to your legs. In particular, this condition involves the patient's right iliac artery, which carries blood to their right leg, and their left iliac vein, which brings blood out of their left leg toward their heart. The right iliac artery squeezes, or pinches, the left iliac vein when they cross each other in the patient's pelvis. Because of that pressure, blood cannot flow as freely through the left iliac vein.

Patients suffering from May-Thurner syndrome are more likely to get a deep vein thrombosis (“DVT”) in their left leg. A DVT is a serious type of blood clot. Not only does a DVT block blood flow in the patient's leg, but it may break off and cause a clot in the patient's lung (i.e., a pulmonary embolism).

Recently, May-Thurner syndrome has been treated by placing a venous stent within the pinched left iliac vein, to thereby open up the left iliac vein. However, this intervention requires placement of permanent implant in the patient.

### SUMMARY

In one embodiment, an endovascular device includes a catheter defining a lumen; a needle extending within the lumen of the catheter and being movable within the catheter, the needle defining a lumen and having a distal needle tip, wherein distal movement of the needle advances the distal needle tip out of the catheter and proximal movement of the needle retracts the distal needle tip within the lumen of the catheter; and a shaving mechanism at least partially arranged within the lumen of the needle, the shaving mechanism having an abrasive burr positioned proximate the distal needle tip, wherein the abrasive burr is rotatable relative to the needle upon activation of the shaving mechanism.

In another embodiment, an endovascular device includes a catheter defining a lumen; a needle extending within the lumen of the catheter having a distal needle tip and being movable within the catheter between retracted and extended positions; and a shaving mechanism at least partially arranged within a lumen of the needle, the shaving mechanism having an abrasive burr positioned proximate the distal needle tip, the abrasive burr being rotatable relative to the needle upon activation of the shaving mechanism; wherein, in the retracted position, the abrasive burr is located within the lumen of the catheter and, in the extended position, the abrasive burr is located outside the lumen of the catheter.

In yet another embodiment, an endovascular device includes a catheter defining a lumen, the catheter includes a window on a sidewall of the catheter positioned proximate a distal end of the catheter; a needle extending within the lumen of the catheter and being movable within the catheter, the needle defining a lumen and having a distal needle tip, wherein distal movement of the needle advances the distal needle tip through the window and out of the catheter, and proximal movement of the needle retracts the distal needle tip through the window and within the lumen of the catheter; a shaving mechanism at least partially arranged within the lumen of the needle, the shaving mechanism having an abrasive burr positioned proximate the distal needle tip, wherein the abrasive burr is rotatable relative to the needle upon activation of the shaving mechanism; and a slider mechanism operably connected to the needle for advancing the needle into an extended position, where the needle extends through the window and out of the catheter, the slider mechanism comprising an actuator operably connected to the abrasive burr for causing rotation of the abrasive burr.

These and additional features provided by the embodiments described herein will be more fully understood in view of the following detailed description, in conjunction with the drawings.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a side view of an endovascular device according to one or more embodiments of the present disclosure.

FIG. 2 is a perspective view of a distal end of the endovascular device of FIG. 1, wherein a needle is in a retracted position within the catheter.

FIG. 3 is a top view of a handle utilizable with the device of FIGS. 1-2, according to one or more embodiments of the present disclosure.

FIG. 4 illustrates various exemplary embodiments of an abrasive burr of the device of FIGS. 1-2, according to one or more embodiments of the present disclosure.

### DETAILED DESCRIPTION

The embodiments described herein provide an endovascular device including a catheter and a needle with built in shaver that may be advanced or extended from the catheter. The catheter may be a polymer catheter having a distal window through which the needle may be extended or retracted. The needle may be a curved heat set super-elastic Nitinol needle. The device may include a handle with a slider mechanism configured to advance the needle out of the distal window of the catheter. The shaver may be driven by an actuator provided in the handle. However, the shaver may be driven by an external actuator, such as an independent motor unit that is external of the handle of the device. [The needle may have platinum marker bands, bands of radiopaque material, or other indicators for aiding visualization of the device during a procedure. In embodiments, rotational indicators may be provided on the needle for rotationally aligning and orienting the shaver during a procedure. In some examples, the device may be configured to apply aspiration within a lumen of the needle so as to remove any boney debris removed by the shaver. By providing a shaver on a needle that is advanceable from a catheter, it is possible to remove portions of the bone structure via an endovascular approach. Moreover, utilization of the presently described endovascular device provides an alternative to placing a stent with the patient. Thus, using the presently described endovascular device prevents occurrence of in-stent restenosis/occlusion, thereby reducing rate of re-intervention. Furthermore, utilization of the presently described endovascular device may allow the patient to retain venous valve function that may otherwise be obstructed by a stent where utilized.

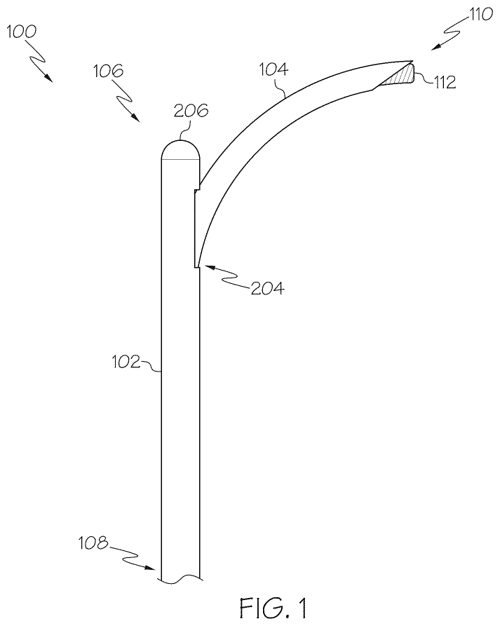

FIG. 1 is a side view of an endovascular device 100 (hereinafter, the device 100), according to one or more embodiments of the present disclosure. The device 100 includes a catheter 102 and a needle 104. As shown, the catheter 102 has a distal end 106 and a proximal end 108. It will be appreciated, however, that FIG. 1 is a partial view of the device 100 depicting just a distal most portion thereof, and that, while not fully illustrated in FIG. 1, the proximal end of the device 100 may extend substantially proximal beyond the proximal end 108 of the catheter 102, out of the patient.

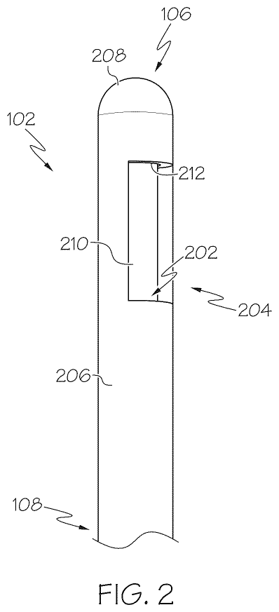

As hereinafter described, the needle 104 is movable within the catheter 102 between retracted and extended positions. FIG. 1 illustrates the device 100 wherein the needle 104 has been advanced into the extended position. FIG. 2 illustrates the device 100, wherein the needle 104 has been moved proximally (retracted) within the catheter 102 into the retracted position. In addition, FIG. 2 illustrates the catheter 102, according to one or more embodiments of the present disclosure. As shown, the catheter 102 has an internal lumen 202 within which the needle 104 (FIG. 1) is slidably provided. The catheter 102 also includes a window 204 provided in a sidewall 206 of the catheter 102 that is in fluid communication with the internal lumen 202 of the catheter 102. In the illustrated example, the window 204 is provided proximate the distal end 106 of the catheter 102. However, the window 204 may be provided elsewhere on the catheter 102, for example, at a location on the catheter 102 more proximal than the window 204 illustrated in FIG. 1, or on a distal tip 208 of the catheter 102, etc. The catheter 102 may be manufactured from various materials utilized in catheter applications, for example, the catheter 102 may comprise a polymer material.

Referring back to FIG. 1, the needle 104 extends within the lumen 202 of the catheter 102 and is movable or slidable within the internal lumen 202 of the catheter 102. In particular, the needle 104 may move distally or proximally within the internal lumen 202 of the catheter 102. Thus, the needle 104 may be advanced, or moved distally, such that a distal tip 110 of the needle 104 is advanced out of the catheter 102. Also, the needle 104 may be retracted, or moved proximally, such that the distal tip 110 of the needle retracted back inside of the catheter 102. In the illustrated example, distal movement of the needle 104 advances the distal tip 110 of the needle 104 through the window 204 and out of the lumen 202 of the catheter 102, such that the needle 104 is in an extended position; whereas, proximal movement of the needle 104 retracts the distal tip 110 of the needle 104 through the window 204 and into the lumen 202 of the catheter 102, such that the needle 104 is in extended retracted position. As hereinafter described, the distal tip 110 of the needle 104 is designed to pierce and then pass through a vein or artery wall so as to be located proximate to a bone structure inside the patient. Thus, the needle 104 may be distally advanced substantially beyond the distance illustrated in FIG. 1 so as to be positioned proximate the patient's bone.

In some examples, the needle 104 is a curved, heat set, super-elastic Nitinol needle. In some examples, a ramp is provided within the lumen 202 of the catheter 102 to facilitate directing the needle 104 out of the lumen 202 as it is advanced therefrom. For example, a ramp may be formed on an interior wall 210 of the lumen 202, opposite the interior side of the window 204, and the ramp may approach the window 204 in the distal direction such that the ramp eventually meets a distal edge 212 of the window 204.

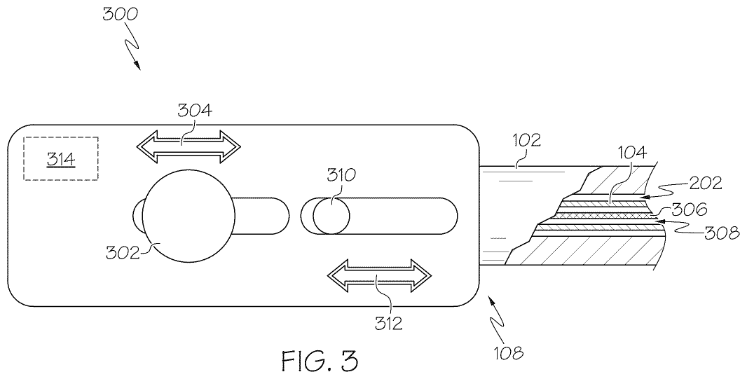

The device 100 further includes a shaving mechanism at least partially arranged within the needle. In particular, the needle 104 is hollow and includes a lumen (obscured from view, see FIG. 3), and the shaving mechanism includes an abrasive burr 112 operably provided within the distal tip 110 of the needle 104. The abrasive burr 112 is rotatable relative to the needle 104 upon activation of the shaving mechanism and, upon such activation, the abrasive burr 112 is operable to shave or chip bone material and thereby form a pocket or other recess in a boney structure.

In the illustrated example, the abrasive burr 112 is illustrated as being partially extended from the lumen of the needle 104, such that the abrasive burr 112 is at least partially exposed and able to abrade or grind bone with which it may be placed in contact with as hereinafter described. However, the abrasive burr 112 may be further distally advanced out of the lumen of the needle 104 beyond what is illustrated in FIG. 1 and, moreover, abrasive burr 112 may be further proximally retracted inside the lumen of the needle 104 beyond what is illustrated in FIG. 1. In embodiments, the abrasive burr 112 may be further distally advanced out of the lumen of the needle 104 to access a bone structure as needed in a procedure. Thus, an outer diameter of the abrasive burr 112 is smaller than an inner diameter of the lumen of the needle 104 so that the abrasive burr 112 may slide in and out of the needle 104. For example, when puncturing the artery or vein, the abrasive burr 112 may be sheathed inside the lumen of the needle 104 so that the abrasive burr 112 would not interact with the puncture.

The shaving mechanism includes an actuator (not shown) configured to cause rotation of the abrasive burr 112. The actuator may be provided within a handle of the device 100 and may be configured to cause rotation of a flexible shaft. The flexible shaft may extend through the lumen of the needle 104 and couple the abrasive burr 112 to the actuator such that actuation of the actuator rotates the flexible shaft, which in turn causes rotation of the abrasive burr 112. The flexible shaft coupled to the abrasive burr 112 may comprise a high torque flexible drive cable or cable tube.

FIG. 3 illustrates a handle 300 provided at the proximal end 108 of the device 100, according to one or more embodiments of the present disclosure. In the illustrated example, the handle 300 includes a slider mechanism 302 operatively coupled to the needle 104 for moving the needle 104 within the 202 of the catheter 102. Here, the slider mechanism 302 is movable in the proximal or distal direction, as indicated by arrow 304, to thereby move the needle 104 connected thereto in the corresponding proximal or distal direction. In particular, the slider mechanism 302 may be manipulated to distally move the needle 104 into the extended position, such that the distal tip 110 of the needle 104 advances through the window 204 and out of the lumen 202 of the catheter 102 as shown in FIG. 1. In addition, the slider mechanism 302 may be manipulated to proximally move the needle 104 into the retracted position, such that the distal tip 110 of the needle 104 is fully retracted within the lumen 202 of the catheter 102 as shown in FIG. 2.

The shaving mechanism includes an actuator 314, a flexible shaft 306, and the abrasive burr 112. The flexible shaft 306 is coupled to the abrasive burr 112, such that rotation of the flexible shaft 306 correspondingly rotates the abrasive burr 112. The flexible shaft 306 is also operatively coupled to the actuator 314, and the actuator 314 is configured to cause rotation of the flexible shaft 306 upon activation of the actuator 314. Thus, activation of the actuator 314 causes rotation of the abrasive burr 112.

The actuator 314 may be provided within the handle 300 of the device 100. The flexible shaft 306 may extend through a lumen 308 of the needle 104 and couple the abrasive burr 112, which is located at the distal end 106, to the actuator 314, which is located at the proximal end 108, such that actuation of the actuator 314 rotates the flexible shaft, which in turn causes rotation of the abrasive burr 112. In embodiments, the actuator may be an external actuator located outside (or independent from) the handle of the device 100, in which case the external actuator is operably coupled to flexible shaft such that actuation of the external actuator rotates the flexible shaft, which in turn causes rotation of the abrasive burr 112. The external actuator may be coupled to the flexible shaft in a variety of manners, for example, the flexible shaft may be operably coupled to an internal drive mechanism of the external actuator and the flexible shaft may extend therefrom to the device 100, where the flexible shaft is then routed through the lumen of the needle 104 and attached to the abrasive burr 112 at a distal end of the flexible shaft.

In the illustrated example, the handle 300 also includes another slider mechanism 310 operatively coupled to the abrasive burr 112, via the flexible shaft 306, for moving the abrasive burr 112 relative to the distal tip 110 of the needle 104. Here, the slider mechanism 310 is movable in the proximal or distal direction, as indicated by arrow 312, to thereby move the flexible shaft 306 and the abrasive burr 112 carried thereon in the corresponding proximal or distal direction. In particular, the slider mechanism 310 may be manipulated to distally move the abrasive burr 112 into a position where it is sufficiently exposed and operable to shave a boney structure. In some examples, the abrasive burr 112 may be extended at least partially beyond the distal tip 110 of the needle 104, whereas in other examples, the distal tip 110 of the needle 104 is distal of the entirety of the abrasive burr 112 when the abrasive burr 112 is distally moved into the exposed position. Also, the slider mechanism 310 may be manipulated to proximally move the flexible shaft 306 to thereby retract the abrasive burr 112 within the lumen 308 of the needle 104. In some examples, the abrasive burr 112 may be fully retracted within the lumen 308 of the needle 104 such that it is not visible, whereas in other examples, the abrasive burr 112 may be partially retracted within the lumen 308 of the needle 104 such that it is partially visible. In even other examples, the abrasive burr 112 is sufficiently retracted within the lumen 308 of the needle 104 a sufficient amount such that the distal tip 110 of the needle 104 is operable to pierce a vein or artery wall without the abrasive burr 112 adversely affecting such piercing action. For example, as mentioned above, when puncturing the artery or vein wall the abrasive burr 112 may be at least partially retracted within the lumen 308 of the needle 104 to an extent such that the abrasive burr 112 would not interact with the artery or vein wall being punctured and then, after puncturing, the abrasive burr 112 may be advanced distally out of the lumen 308 of the needle 104. In some examples, the shaving mechanism is configured to cause actuation of the actuator 314, thereby rotating the abrasive burr 112, upon distal extension/advancement of the flexible shaft 306 and the abrasive burr 112 via the slider mechanism 310. Thus, the slider mechanism 310 may cause rotation of the abrasive burr 112 as it is being manipulated to advance the abrasive burr 112. In other examples, the shaving mechanism includes a separate means for causing rotation of the abrasive burr 112 independent of the slider mechanism 310.

When placing the needle 104, fluoroscopy and/or imaging may be utilized to ensure the abrasive burr 112 travels the appropriate distance. In some embodiments, the needle 104 may have platinum marker bands, bands of radiopaque material, or other indicators for aiding visualization of the device 100 during a procedure. In embodiments, the needle may also or instead include rotational indicators may be provided on the needle for rotationally aligning and orienting the shaver during a procedure.

In some embodiments, the device 100 could provide vacuum aspiration. For example, vacuum aspiration may be provided within the lumen 308 of the needle 104 to help remove with bone debris cut away by the abrasive burr 112. In this manner, no bone debris would travel upstream in the arterial/venous system, because the needle 104 would be poking outside the artery/vein wall and thus operating in the space between artery/vein and bone structure such that any bone debris would remain there or be aspirated out of the patient.

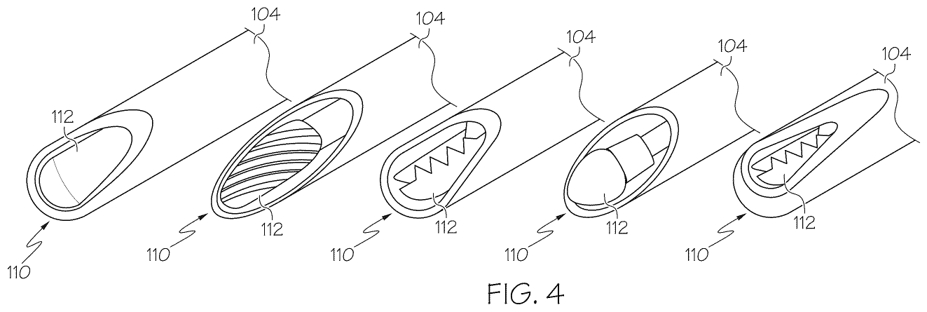

As illustrated in FIG. 4, the abrasive burr 112 provided in the distal tip 110 of the needle 104 may have various configurations cutting geometries. In particular, FIG. 4 illustrates various exemplary cutting geometries of the abrasive burr 112. For example, the abrasive burr 112 may comprise a cylindrically shaped head, a partially cylindrically shaped head with one or more flat surfaces on a side thereof, an ellipse shaped head, a sphere shaped head or a partially spherical shaped head, a tapered shape head, etc. Regardless of head shape, the abrasive burr 112 may have flutes and cutting edges arranged thereon, for example, helical extending flutes and cutting edges. Alternatively, the abrasive burr 112 may include longitudinally or helically extending ridges, or a plurality of teeth provided on an outer surface of the abrasive burr 112, etc.

A procedure utilizing the device 100 described herein would first comprise gaining access to the arterial or venous system, for example, the femoral or popliteal. Then, the procedure would comprise tracking the catheter 102 to the location of compression, for example, using fluoroscopy. Once located at the intervention site, the procedure would comprise advancing the needle 104 out of the catheter 102, for example, via the window 204. Thereafter, the procedure may comprise precise refining of the trajectory of the needle 104, such that the distal tip 110 of the needle 104 is sufficiently positioned next to the bone that needs to be shaved via the abrasive burr 112. Properly positioning the distal tip 110 of the needle 104 helps ensure that subsequent shaving occurs at the desired location, as it is desirable to avoid the spine/bone structures other than the small amount of bone needed to be shaved to relieve the vessel compression. In embodiments, the location of the distal tip 110 of the needle 104 within the artery/vein anterior posterior and lateral fluoroscopic views.] Once final location and trajectory of the needle 104 are confirmed, the procedure may comprise distally advancing the needle 104 and piercing the artery/vein wall with the distal tip 110 of the needle 104, such the needle 104 advances out of the artery/vein, until the distal tip 110 of the needle 104 contacts the bone structure. In some examples, the procedure may then include distally moving the abrasive burr 112 relative to the distal tip 110 to thereby expose the abrasive burr 112. Then, the procedure comprises confirming activating the shaving mechanism and thereby rotating the abrasive burr 112 to thereby chip or shave away bone. During the procedure, the needle 104 may be repositioned as needed. The procedure may be utilized to create or form a slight pocket in the bone, thereby allowing the vein to become decompressed and more circular in nature.

By utilizing an endovascular device comprising a shaver provided on a needle that is advanceable from a catheter, it is possible to remove bone from the via an endovascular approach, and represents an alternative interventional approach than placing a stent with the patient. Thus, using the presently described endovascular device prevents occurrence of in-stent restenosis/occlusion, thereby reducing rate of re-intervention. Furthermore, utilization of the presently described endovascular device may allow the patient to retain venous valve function that may otherwise be obstructed by a stent where utilized.

Embodiments can be described with reference to the following numbered clauses, with certain features laid out in the dependent clauses:

Clause 1: An endovascular device, comprising: a catheter defining a lumen; a needle extending within the lumen of the catheter and being movable within the catheter, the needle defining a lumen and having a distal needle tip, wherein distal movement of the needle advances the distal needle tip out of the catheter and proximal movement of the needle retracts the distal needle tip within the lumen of the catheter; and a shaving mechanism at least partially arranged within the lumen of the needle, the shaving mechanism having an abrasive burr positioned proximate the distal needle tip, wherein the abrasive burr is rotatable relative to the needle upon activation of the shaving mechanism.

Clause 2: the endovascular device of clause 1, wherein the catheter includes a window positioned proximate a distal end of the catheter, wherein advancement of the needle extends the needle outside the catheter through the window.

Clause 3: the endovascular device of any of clauses 1-2 further comprising a first slider mechanism operably connected to the needle for advancing the needle from the catheter.

Clause 4: the endovascular device of any of clauses 1-3 further comprising actuator operably connected to the shaving mechanism for causing rotation of the abrasive burr.

Clause 5: the endovascular device of any of clauses 1-4, further comprising a second slider mechanism configured to distally advance the abrasive burr at least partially beyond the distal needle tip.

Clause 6: the endovascular device of any of clauses 1-2 further comprising a first slider mechanism operably connected to the needle for advancing the needle from the catheter and an actuator operably connected to the shaving mechanism for causing rotation of the abrasive burr.

Clause 7: the endovascular device of clause 6, further comprising a second shaving mechanism configured to distally advance the abrasive burr at least partially beyond the distal needle tip.

Clause 8: the endovascular device of any of clauses 1-7, wherein the needle is configured to apply vacuum aspiration within the lumen of the needle and thereby create suction at the distal needle tip.

Clause 9: the endovascular device of any of clauses 1-8, wherein the needle comprises a curved heat set super-elastic Nitinol needle.

Clause 10: the endovascular device of any of clauses 1-9, wherein the catheter comprises a polymer material.

Clause 11: the endovascular device of any of clauses 1-10, wherein the needle comprises platinum marker bands for aiding its placement.

Clause 12: An endovascular device, comprising: a catheter defining a lumen; a needle extending within the lumen of the catheter having a distal needle tip and being movable within the catheter between retracted and extended positions; a shaving mechanism at least partially arranged within a lumen of the needle, the shaving mechanism having an abrasive burr positioned proximate the distal needle tip, the abrasive burr being rotatable relative to the needle upon activation of the shaving mechanism; and wherein, in the retracted position, the abrasive burr is located within the lumen of the catheter and, in the extended position, the abrasive burr is located outside the lumen of the catheter.

Clause 13: the endovascular device of clause 11, wherein the catheter includes a window positioned proximate a distal end of the catheter and, the extended position, the distal needle tip is extending through the window.

Clause 14: the endovascular device of any of clauses 11-12 further comprising a first slider mechanism operably connected to the needle for advancing the needle out of the catheter.

Clause 15: the endovascular device of any of clauses 11-13 further comprising an actuator operably connected to the shaving mechanism for causing rotation of the abrasive burr.

Clause 16: the endovascular device of any of clauses 11-14, further comprising a second slider mechanism configured to distally advance the abrasive burr at least partially beyond the distal needle tip.

Clause 17: the endovascular device of any of clauses 11-15, wherein the needle is configured to apply vacuum aspiration within the lumen of the needle and thereby create suction at the distal needle tip.

Clause 18: the endovascular device of any of clauses 11-16, wherein the needle comprises a curved heat set super-elastic Nitinol needle.

Clause 19: An endovascular device, comprising: a catheter defining a lumen, the catheter includes a window positioned proximate a distal end of the catheter; a needle extending within the lumen of the catheter and being movable within the catheter, the needle defining a lumen and having a distal needle tip, wherein distal movement of the needle advances the distal needle tip through the window and out of the catheter, and proximal movement of the needle retracts the distal needle tip through the window and within the lumen of the catheter; a shaving mechanism at least partially arranged within the lumen of the needle, the shaving mechanism having an abrasive burr positioned proximate the distal needle tip, wherein the abrasive burr is rotatable relative to the needle upon activation of the shaving mechanism; and a slider mechanism operably connected to the needle for advancing the needle into an extended position, where the needle extends through the window and out of the catheter, the shaving mechanism comprising an actuator operably connected to the abrasive burr for causing rotation of the abrasive burr.

Clause 20: the endovascular device of clause 19, further comprising another slider mechanism configured to distally advance the abrasive burr at least partially beyond the distal needle tip when in the extended position.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

The terms “proximal” and “distal” are defined herein relative to a surgeon utilizing the venous thrombectomy device. The term “proximal” refers to the position of an element closer to the surgeon and the term “distal” refers to the position of an element further away from the surgeon. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

1. An endovascular device, comprising:
a catheter defining a lumen;
a needle extending within the lumen of the catheter and being movable within the catheter, the needle defining a lumen and having a distal needle tip, wherein distal movement of the needle advances the distal needle tip out of the catheter and proximal movement of the needle retracts the distal needle tip within the lumen of the catheter; and
a shaving mechanism at least partially arranged within the lumen of the needle, the shaving mechanism having an abrasive burr positioned proximate the distal needle tip, wherein the abrasive burr is rotatable relative to the needle upon activation of the shaving mechanism.

2. The endovascular device of claim 1, wherein the catheter includes a window positioned proximate a distal end of the catheter, wherein advancement of the needle extends the needle outside the catheter through the window.

3. The endovascular device of claim 1, further comprising a first slider mechanism operably connected to the needle for advancing the needle from the catheter.

4. The endovascular device of claim 1, further comprising an actuator operably connected to the shaving mechanism for causing rotation of the abrasive burr.

5. The endovascular device of claim 1, further comprising a second slider mechanism configured to distally advance the abrasive burr at least partially beyond the distal needle tip.

6. The endovascular device of claim 1, further comprising a first slider mechanism operably connected to the needle for advancing the needle from the catheter and an actuator operably connected to the shaving mechanism for causing rotation of the abrasive burr.

7. The endovascular device of claim 6, further comprising a second slider mechanism configured to distally advance the abrasive burr at least partially beyond the distal needle tip.

8. The endovascular device of claim 1, wherein the needle is configured to apply vacuum aspiration within the lumen of the needle and thereby create suction at the distal needle tip.

9. The endovascular device of claim 1, wherein the needle comprises a curved heat set super-elastic Nitinol needle.

10. The endovascular device of claim 1, wherein the catheter comprises a polymer material.

11. The endovascular device of claim 1, wherein the needle comprises platinum marker bands for aiding its placement.

12. An endovascular device, comprising:
a catheter defining a lumen;
a needle extending within the lumen of the catheter having a distal needle tip and being movable within the catheter between retracted and extended positions;
a shaving mechanism at least partially arranged within a lumen of the needle, the shaving mechanism having an abrasive burr positioned proximate the distal needle tip, the abrasive burr being rotatable relative to the needle upon activation of the shaving mechanism; and
wherein, in the retracted position, the abrasive burr is located within the lumen of the catheter and, in the extended position, the abrasive burr is located outside the lumen of the catheter.

13. The endovascular device of claim 12, wherein the catheter includes a window positioned proximate a distal end of the catheter and, the extended position, the distal needle tip is extending through the window.

14. The endovascular device of claim 12, further comprising a first slider mechanism operably connected to the needle for advancing the needle out of the catheter.

15. The endovascular device of claim 12, further comprising an actuator operably connected to the shaving mechanism for causing rotation of the abrasive burr.

16. The endovascular device of claim 12, further comprising a second slider mechanism configured to distally advance the abrasive burr at least partially beyond the distal needle tip.

17. The endovascular device of claim 12, wherein the needle is configured to apply vacuum aspiration within the lumen of the needle and thereby create suction at the distal needle tip.

18. The endovascular device of claim 12, wherein the needle comprises a curved heat set super-elastic Nitinol needle.

19. An endovascular device, comprising:
a catheter defining a lumen, the catheter includes a window on a sidewall of the catheter positioned proximate a distal end of the catheter;
a needle extending within the lumen of the catheter and being movable within the catheter, the needle defining a lumen and having a distal needle tip, wherein distal movement of the needle advances the distal needle tip through the window and out of the catheter, and proximal movement of the needle retracts the distal needle tip through the window and within the lumen of the catheter;
a shaving mechanism at least partially arranged within the lumen of the needle, the shaving mechanism having an abrasive burr positioned proximate the distal needle tip, wherein the abrasive burr is rotatable relative to the needle upon activation of the shaving mechanism; and
a slider mechanism operably connected to the needle for advancing the needle into an extended position, where the needle extends through the window and out of the catheter, the slider mechanism comprising an actuator operably connected to the abrasive burr for causing rotation of the abrasive burr.

20. The endovascular device of claim 19, further comprising another slider mechanism configured to distally advance the abrasive burr at least partially beyond the distal needle tip when in the extended position.
